(19) United States
(12) Patent Application Publication
(10) Pub. No.: US 2017/0224532 A1
(43) Pub. Date: Aug. 10, 2017

(54) METERED DOSE EYEDROPPER AND METHOD OF ADMINISTERING A METERED DOSE ONTO THE SURFACE OF THE EYE

(71) Applicant: Bullseye Dropper, LLC, Covington, KY (US)

(72) Inventor: Robert Behan, Covington, KY (US)

(21) Appl. No.: 15/519,207

(22) PCT Filed: Oct. 16, 2015
(86) PCT No.: PCT/US2015/055983

§ 371 Date: Apr. 14, 2017

Related U.S. Application Data
(60) Provisional application No. 62/064,830, filed on Oct. 16, 2014.

(51) Int. Cl. A61F 9/00

(52) U.S. Cl. CPC A61F 9/0008; A61F 9/0026

(57) ABSTRACT

An eyedropper is provided for administering a predetermined volume of liquid onto the surface of an eye. The eyedropper includes a body having a nozzle in fluid communication with the internal cavity of the reservoir body. Projecting from an inner surface of the reservoir body are a first projecting portion having a first contact surface and a second projecting portion having a second contact surface. The first contact surface and the second contact surface are disposed opposite from one another and are spaced apart from one another. In use, the first and second contact surfaces limit the displacement volume of the internal cavity when the eyedropper is squeezed thereby limiting the volume of liquid expelled from the nozzle.

### CROSS-REFERENCE TO RELATED APPLICATIONS

This application claims the benefit of U.S. Patent Application No. 62/064,830 filed on Oct. 16, 2014, the disclosures of which are expressly incorporated by reference herein in their entirety.

### FIELD

Aspects of the invention are generally related to devices and methods for administering a metered dose of liquid from a container. More particularly, aspects of the invention are related to an eyedropper having a structure allowing for the administration of a metered dose of liquid onto the surface of a subject's eye and to methods of using such an eyedropper.

### BACKGROUND

Presently, eye drops come in a variety of dropper bottle shapes and sizes to administer a variety of drops into the eyes of a subject. Those devices typically have a bottle base with a nozzle attached thereto for administering the drops. Although the details may vary, the majority of eye drop bottles effectively have similar shapes, styles, and uses. Products such as Visine®, Clear Eyes® and the like, lead the field with eye drop bottles that have been used for years but are, however, antiquated.

Administration of drops into the eye is not easy with current eyedropper bottles. Most eye drop bottles currently in use are available in sizes that are able to be held in between the thumb and forefinger. The bottle is squeezed so that one or more drops are expelled from the bottle when aimed in the general direction of the eye after the head is tilted back resulting in a drop that hopefully makes its way onto the surface of the eye. If the subject administering the drop has shaky hands, such as in many elderly patients, or the subject having drops administered into his or her eyes cannot hold his head still, such as with a small child, this process can be even more difficult. Thus, presently available eyedropper bottles often result in inaccurate placement of the drop resulting in drops landing on other parts of the body surrounding the eye, on clothes, or elsewhere, which could affect the areas the drops contact.

In addition, presently available eyedropper bottles can also result in multiple drops coming out instead of just one, which can cause waste. The ability to carefully squeeze the bottle to limit the volume of liquid dispensed from the nozzle while looking up at it to keep it on target is strenuous and then attempting to look away at the right moment so that the eye drop does not hit the pupil is difficult at best. Many users need two hands to administer eye drops to themselves because they operate the eyedropper bottle with one hand while using their other hand to hold open their eyelids to assist them in administering drops onto the surface of the eye.

Previous efforts to improve eyedropper bottles have included attaching various structures to conventional bottles to limit the volume of liquid dispensed during use. These added structures present additional shortcomings. Some devices utilize complicated mechanical structures to deliver the drop from the bottle onto the surface of the eye. Other devices require assembly onto the eyedropper bottle before use, which means that the user must keep up with the additional components.

Today's eyedropper bottles used with over the counter and prescription medications cause anxiety and or frustration to the end user and result in significant waste as extra drops of medicated eye drops are frequently administered when only a single drop is needed. This can be especially costly with eye medications, such as glaucoma drugs and antibiotics, which are provided in limited volumes and at great cost to the patient. A solution to the waste of today's eyedropper bottles is needed and is hereby addressed with this invention.

### SUMMARY

Aspects of the invention are directed to an eyedropper that makes it easy, safe, and effective to administer a predetermined volume of liquid to the eye. The embodiments of the invention (1) can administer a predetermined volume of liquid, such as one drop at a time, (2) are less likely to leak around the nozzle, (3) are stabilized by contacting the face on opposite sides of the eye socket, such as above and below or medial and lateral the eye socket, so when the reservoir body of the eyedropper is squeezed between fingers it accurately administers a predetermined volume of liquid onto the surface of the eye, (4) can administer a predetermined volume of liquid exactly where intended, (5) can be operated with a single hand, (6) can decrease neck tilt, (7) can assist in pulling the skin below or above the eye, away from the eye socket to assist with maintaining the eyelids in an open position with one hand, (8) can reduce anxiety and or frustration in the end user, and (9) can create a satisfied user. More particularly, when in use to administer drops of varying makeup or solution, embodiments of the invention stabilize the eyedropper and some embodiments can administer a predetermined volume of liquid (or single drop at a time), decrease head tilt, and allow for one handed operation while decreasing anxiety of the user.

Accordingly, an eyedropper is described for administering a predetermined volume of liquid onto the surface of an eye having a body having a reservoir body with an inner surface defining an internal cavity, and a nozzle in fluid communication with the internal cavity. The inner surface has a first contact surface on a first projecting portion and a second contact surface. The first contact surface is opposed to and spaced apart from the second contact surface and is configured to limit the displacement of the volume of the internal cavity when the body is squeezed to an amount sufficient to expel a predetermined volume of liquid from the nozzle.

Another aspect of the invention is directed to a nozzle structure that reduces the likelihood of leaking around the nozzle as compared to other nozzle designs. This design is particularly useful with bottles made of highly flexible materials.

### BRIEF DESCRIPTION OF THE DRAWINGS

The accompanying drawings, which are incorporated in and constitute a part of this specification, illustrate various embodiments of the invention and, together with a general description of the invention given above and the detailed description of the embodiments given below, serve to explain the embodiments of the invention.

FIG. 1 illustrates the use of an embodiment of an eyedropper in accordance with embodiments of the invention.

FIG. 2 is a longitudinal cross-sectional side view of an exemplary embodiment of an eyedropper in accordance with embodiments of the invention.

FIG. 3 is a cross-sectional view of the exemplary embodiment of the eyedropper in FIG. 2 taken along line 3-3.

FIG. 4 is a cross-sectional view of the exemplary embodiment of the eyedropper in FIG. 2 taken along line 4-4.

FIG. 4A is an enlarged cross-sectional view of the exemplary embodiment of the eyedropper in FIG. 2 of encircled area 4A.

FIG. 5 is a front elevation view partially broken away of an alternative exemplary embodiment of an eyedropper in accordance with embodiments of the invention.

### DETAILED DESCRIPTION

An aspect of the invention is directed to an eyedropper capable of easily and repeatedly dispensing a predetermined volume of liquid. Embodiments of the eyedropper dispense the predetermined volume of liquid by limiting the displacement of the internal volume of the bottle that occurs when a user squeezes the side walls of the bottle. Embodiments of the eyedropper includes one or more pairs of surfaces extending from opposite sides of the inner surface that defines the cavity of the eyedropper that contact each other when the bottle is squeezed to limit the volume of internal displacement that occurs with each squeeze of the bottle.

Further aspects of the invention are directed to eyedroppers capable of easily and repeatedly delivering a predetermined volume of liquid onto the surface of the eye utilizing the general concept described above.

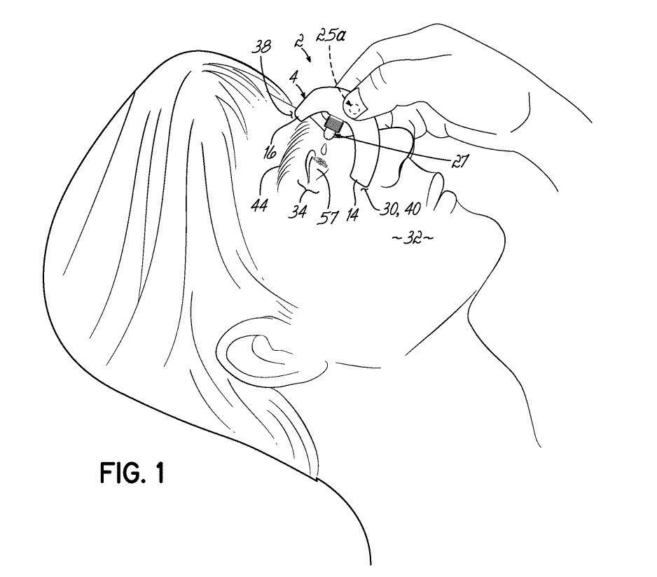

With reference to FIGS. 1 to 4A, embodiments of the invention are directed to an eyedropper 2 having a body 4 with a first leg 6 and a second leg 8. The first leg 6 includes a first foot 14 located at one end of the body 4 and the second leg includes a second foot 16 at the opposite end of the body 4. The body 4 also includes a reservoir body 18 intermediate to the first and second feet 14, 16. The reservoir body 18 includes an outer surface 20 and an inner surface 22 defining a portion of an internal cavity 24. The inner surface 22 of the reservoir body 18 also includes a first projecting portion 25a and an oppositely disposed and spaced apart second projecting portion 25b extending into the internal cavity 24. The eyedropper 2 also includes a nozzle 27 projecting from the outer surface 20 of the reservoir body 18 between the first and second feet 14, 16. The nozzle 27 is in fluid communication with the internal cavity 24.

The first projecting portion 25a has a first contact surface 26a that is spaced apart from and adjacent to a second contact surface 26b on the second projecting portion 25b. When the eyedropper 2 is squeezed, the sidewalls of the eyedropper 2 deflect inward thereby decreasing the internal volume in the eyedropper 2 to cause liquid in the internal cavity 24 above the nozzle 27 to be expelled from the nozzle 27. The volume of liquid expelled from the nozzle 27 is related to the decrease in the volume of the internal cavity 24 caused when the sidewalls of the eyedropper 2 are squeezed. The volume of the internal cavity 24 that is displaced when the eyedropper 2 is squeezed is determined, at least in part, by the volume of the first and second projecting portions 25a, 25b and the distance between the first and second contact surfaces 26a, 26b. The volume of the first and second projecting portions 25a, 25b and the distance between the contact surfaces 26a, 26b are sufficient to allow for the displacement of a volume of the internal cavity 24 sufficient to expel the desired volume of liquid from the nozzle 27. In an embodiment, displaced volume of the internal cavity 24 is greater, i.e., about 15% to about 75% greater and preferably about 25% to about 50% greater, than the desired volume of liquid to be expelled from the bottle so as to provide a force sufficient to break the surface tension between the volume of liquid and the nozzle 27.

The first foot 14 has a first end surface 28 configured to contact a first area 30 of a subject's face 32 adjacent the subject's eye socket 34 (FIG. 1). The second foot 16 has a second end surface 36 configured to contact a second area 38 of the subject's face 32 on the opposite side of the eye socket 34. In an embodiment, the first foot 14 is configured to contact the subject's face 32 below the eye socket 34, such as on or near the cheek 40, and the second foot 16 is configured to contact the subject's face 32 at a location above the eye socket 34, such as on or near the eyebrow 44 (FIG. 1). The first foot 14 may alternatively contact a first area 30 on the subject's face generally lateral to the eye socket 34, such as on or near the temple area, and the second foot 16 may contact a second area medial to the eye socket 34, such as on or near the bridge of the nose.

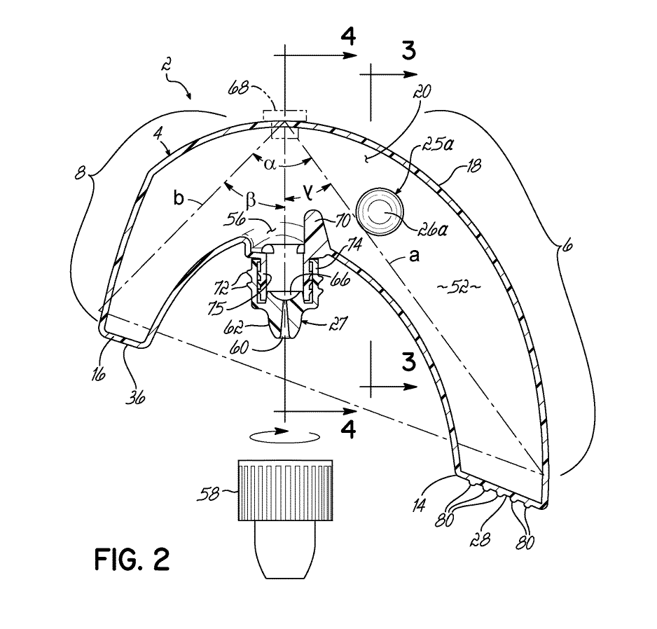

The relationship between the first foot 14, and the second foot 16, which may be defined at least in part by the angle between the first leg 6, and the second leg 8 may be defined relative to a first axis a associated with the first leg 6, a second axis b associated with the second leg, and a third axis c associated with the nozzle 27. The third axis c is defined by the longitudinal axis of the nozzle 27 and projects along a center line through the tip 62 of the nozzle 27 to a point that intersects the outer surface 20 of the body 4 that overlies the nozzle 27. The first axis a is defined by a line extending through the length of the first leg 6 from the point along the outer surface 20 of the first leg 6 or first foot 14 that is the greatest distance in a straight line from the intersection of the third axis c with the outer surface 20 of the body 4. The second axis b is defined by a line extending through the length of the second leg 8 from the point along the outer surface 20 of the second leg 8 or second foot 16 that is the greatest distance in a straight line from the intersection of the third axis c with the outer surface 20 of the body 4. Thus, the first, second, and third axes intersect at a point on the outer surface 20 opposite the nozzle 27 and may be used to define the angular relationship between the first and second legs 6, 8 and the nozzle 27. The intersection of the first axis a with the second axis b forms an angle a that is less than 180 degrees, and preferably is in a range from about 45 degrees to about 175 degrees, and more preferably from about 90 degrees to about 160 degrees (FIG. 2). The intersection of the first leg 6 with the second leg 8 may be defined by the intersection a first axis a with the second axis b and the third axis c. The angle a formed by the intersection of the first axis a with the second axis b is bisected by the third axis c. The angle β between the second axis b and the third axis c is typically less than or equal to the angle y between the first axis a and the third axis c. Preferably, the angle β is less than the angle y.

In an embodiment of the invention, the nozzle is closer to one of the first or second feet 14, 16 than it is to the other foot. For example, in the exemplary embodiments shown in FIGS. 1 to 4A, the first axis a of the first leg 6 has a first length and the second axis b of the second length that is less than the first length of the first axis a. The first length of the first axis a may be about 1.1 to about 2 times the second length of the second axis b, or the first length may be about 1.2 to about 1.8 times the second length, or the first length may be about 1.3 to about 1.7 times second length. In another embodiment of the invention, the nozzle 27 is equidistant between the first and second feet 14, 16. In such an embodiment, the first length of the first axis a is approximately equal to the second length of the second axis b.

Another aspect of the invention is the significantly improved stability conferred by the relationship between the height of the eyedropper 2 and the distance between the first 14 and second 16 feet. A distance d between the first foot 14 and the second foot 16 is measured from the outer most edge of the eyedropper as shown in FIG. 2. The height of the eyedropper is the vertical distance between a first plane that is tangential to both the first and second feet and a second plane that is tangential to a point along the outer surface of the body 4 that is parallel to the first plane and that is the greatest vertical distance from the first plane. The distance d is at least equal to the height h of the eyedropper, and preferably the distance d is greater than the height h. In exemplary embodiments, the distance d between the first and second feet of the eyedropper 2 may range from about 4.5 inches to about 2.5 inches. Preferred exemplary embodiments have a distance d of about 4.5 inches, or about 3.1 inches, or about 2.5 inches. The height h of the eyedropper 2 is less than or equal to the distance d and can range from about 1.5 inches to about 2.5 inches. In preferred exemplary embodiments, the height h is about 2.5 inches or about 2 inches, or about 1.5 inches. The ratio of the distance d to height h is at least 1 and preferably greater than 1. The ratio of the distance d to height h may range between about 1 to about 3 or about 1.1 to about 3 or about 1.5 to about 3.

The reservoir body 4 has a width w that is perpendicular to the length of the body along the first and second axes a, b. In an exemplary embodiment, the width w can range from about ½ inch to about 1 ½ inches. In another exemplary embodiment, the width w can range from about ½ inch to about 1 ¼ inches. In another exemplary embodiment, the width w can range from about ⅞ of an inch to about 1 inch.

The reservoir body has an internal cavity 24 is defined by its inner surface 22. In the exemplary embodiment illustrated in FIG. 2, the internal cavity 24 extends from the first foot 14 through the first leg 6 and through the second leg 8 to the second foot 16. In this embodiment, the reservoir body 18 is generally continuous with the first and second legs 6, 8 from the first foot 14 to the second foot 16. This structure provides a reservoir body with a relatively large volume for storing the liquid to be dispensed onto the surface of the eye while maintaining the desired relationship between the height of the eyedropper 2 and the distance d between the first and second feet 14, 16.

In an alternative embodiment, reservoir body does not extend through one or both of the first and second legs toward one or both of the first and second feet, i.e., that at least one of the first or second feet are separated from the reservoir body a solid portion of the leg or a portion of the leg into which the liquid to be dispensed cannot pass. For example, the reservoir body may not extend a significant distance into either of the first leg or the second leg. Instead, the reservoir body may be coupled to the first foot by a solid first leg and may be coupled to the second foot by a solid second leg. While the first and second legs are described in this exemplary embodiment as solid, they could also be hollow or have openings along their length. In a further alternative embodiment, the reservoir body may extend partially into one or both of the first and second legs.

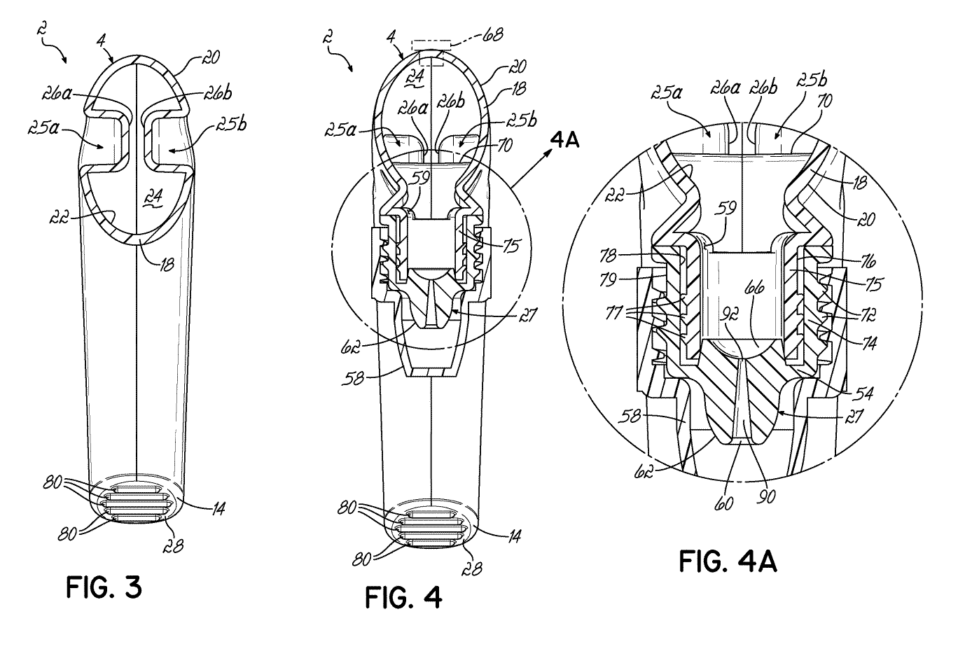

In the exemplary embodiment shown in FIGS. 2 and 3, the internal cavity 24 of the reservoir body 18 includes a reservoir portion 52 nearer the first foot 14 in the first leg 6 and a liquid administering portion 56 located adjacent the nozzle 27. The reservoir portion 52 is stores a volume of liquid whereas the liquid administering portion 56 allows for a volume of liquid to be located above dispensed the nozzle 27 so that a predetermined volume of liquid from the liquid in the liquid administering portion 56 may be dispensed from the nozzle 27. In another embodiment, the liquid stored in the reservoir body 18 may fill both the reservoir portion 52 and the liquid administering portion 56 or may fill just the reservoir portion 52.

The inner surface 22 of the reservoir body 18 presents a relatively smooth surface over which the liquid to be administered may flow toward the nozzle 27. Preferably, the inner surface 22 near the nozzle 27 includes a funnel-shaped portion 59 that leads toward the nozzle 27. The funnel-shaped portion 59 may present a curved surface or a frustoconical surface. The funnel-shaped portion 59 increases the ease with which the user of the eyedropper 2 can utilize the entire contents of the eyedropper because the funnel-shaped portion 59 directs droplets of liquid to the nozzle 27. In an embodiment, the inner surface includes a raised projection, referred to herein as a drop stopper 70, adjacent to the funnel-shaped portion 59 on a side toward at least one of the first or second feet 14, 16 to direct liquid toward the nozzle 27 (FIG. 2). The drop stopper 70 has a height sufficient to direct liquid into the funnel-shaped portion 59 as the eyedropper 2 is being rocked back and forth.

The nozzle 27 projects from the outer surface 20 of the reservoir body 18 and is located between the first and second feet 14, 16. The nozzle 27 includes a channel 64 passing between the opening 60 in the tip 62 of the nozzle 27 to the base 54 of the nozzle 27 that allows for a liquid to be administered to the surface of the eye from the internal cavity 24. The nozzle 27 is coupled to the reservoir body 18 so as to project downwardly from the eyedropper 2 toward the eye when the first and second feet 14, 16 are in contact with the face 32 of the subject (FIG. 1). The tip 62 of the nozzle 27 is situated relative to the first and second feet 14, 16 such that the tip 62 is less likely to contact the eye 57. To accomplish this, the tip 62 of the nozzle 27 does not extend through a plane that is tangential to both the first and second feet 14, 16.

In the exemplary embodiment, the base 54 of the nozzle 27 may include a flange 74 projecting from the periphery of the base. The flange 74 of the nozzle 27 receives a correspondingly shaped and sized nozzle portion 75 extending from reservoir body 18. The outer surface 76 of the nozzle portion 75 of the reservoir body 18 may include one or more annular rings 77. The one or more annular rings 77 may engage the inner surface 78 of the flange 74 and form a fluid tight seal and prevent leakage of liquid from the reservoir body 18. The external surface 79 of the flange 74 nozzle 27 may optionally include a structure for engaging and retaining a cap 58 over the nozzle 27, such as threads 72 for engaging threads on an internal surface of the nozzle cap 58.

The reservoir body 18 is ultimately filled with a liquid prior to use. In embodiment, the reservoir body 18 may be filled through the nozzle 27 or through the opening in the nozzle portion 75 of the reservoir body 18 onto which the nozzle 27 is placed during manufacturing. The reservoir body 18 may optionally include an opening 61 into internal cavity 24 that may be used for filling the internal cavity with a liquid to be administered onto the surface of the eye. The opening 61 may be located along the outer surface 20, such as in the outer curved surface opposite the nozzle 27 as shown in FIG. 2, or may be located on or near the first and second feet 14, 16, or along a side of the reservoir body 4. The opening 61 may be closed such as with a cap 68 or may be blocked with a septum that may be punctured such as with a needle for filling with a syringe.

The base 54 of the nozzle 27 nozzle portion 75 extending from reservoir body 18 may form a nozzle reservoir that is coextensive with the reservoir portion 52. In embodiments of the invention, the nozzle reservoir optionally includes an indentation 66 formed in the base 54 of the 27. The nozzle reservoir may have a predetermined volume to assist with administering a predetermined volume liquid through the channel 64 and opening 60 of the nozzle 27. In the alternative, the volume of the nozzle reservoir may hold a volume of liquid sufficient to administer a plurality of drops of liquid. In some embodiments, limiting the volume of liquid above the nozzle reservoir improves the accuracy of the predetermined volume of liquid dispensed through the nozzle 27. For example, limiting the volume of liquid in the nozzle reservoir will limit the amount of pressure from the liquid column above the nozzle 27 pushing down on the liquid to be dispensed onto the surface of the eye 57.

In an embodiment, the volume of the nozzle reservoir may be, for example, in a range from about 5 microliters to about 50 microliters. In another embodiment, the volume of the nozzle reservoir may be in a range from about 5 microliters to about 35 microliters. In another embodiment, the volume of the nozzle reservoir may be in a range from about 15 microliters to about 50 microliters. In another embodiment, the volume of the nozzle reservoir may be in a range from about 25 microliters to about 50 microliters. In another embodiment, the volume of the nozzle reservoir may be in a range from about 25 microliters to about 35 microliters. In an embodiment, the volume of the nozzle reservoir may be about 15 microliters.

During use, the nozzle reservoir is filled by tilting the eyedropper 2 such that liquid fills the nozzle reservoir and then tilting the eyedropper so that the excess liquid falls into the reservoir portion 52 of the internal cavity 24 of the eyedropper 2. The optional drop stopper 70 directs the liquid into the nozzle reservoir. When the nozzle reservoir is full, the eyedropper is considered into be loaded. The loaded eyedropper 2 is then positioned over the eye with the first and second feet 14, 16 contacting the subject's face on opposite sides of the eye socket (FIG. 1). Once positioned, the subject applies pressure to the outer surface 20 of the reservoir body 4 sufficient to bring together the contact surfaces 26b, 26b of the first and second projecting portions 25a, 25b thereby causing a temporary decrease in the volume of the internal cavity 24 and expelling a predetermined volume of liquid from the nozzle 27. Thus, embodiments of the eyedropper 2 may accurately administer predetermined volumes of liquid onto the surface of the eye. A similar process is used to administer drops from embodiments that do not utilize a nozzle reservoir for administering a predetermined volume of liquid. For example, the exemplary embodiment shown in FIG. 2 may be filled with a volume of liquid such that the reservoir portion 52 and the liquid administering portion 56 are both filled with liquid. In that situation, there is no need for loading the optional nozzle reservoir.

Accurately delivering a predetermined volume of liquid to the surface of the eye is advantageous because the capacity of the eye 57 to hold a volume liquid is limited and drops having a volume that is greater than the eye's capacity will overflow the eye. Current eyedropper systems administer drops having a volume that exceed the volumetric capacity of the eye. The excess liquid administered to the eye either passes through the tear duct or is swept onto the face when the subject blinks his or her eye. The first and second projecting portions 25a, 25b allow for drops to be administered at a predetermined volume and can limit the number of drops administered in a single application. In an embodiment, the predetermined volume does not substantially overflow the volumetric capacity of the eye and is in a range of about 5 microliters to about 50 microliters. In another embodiment, the predetermined volume may be in a range from about 5 microliters to about 35 microliters. In another embodiment, the predetermined volume may be in a range from about 5 microliters to about 35 microliters. In another embodiment, the predetermined volume may be in a range from about 10 microliters to about 25 microliters. In another embodiment, the predetermined volume may be in a range from about 5 microliters to about 15 microliters. In another embodiment, the predetermined volume may be about 15 microliters. In an embodiment, a single squeeze of the eyedropper will result in a single drop being administered. An additional advantage of delivering a predetermined volume and limiting the number of drops administered is that medicines that must be administered at a specific dose into the eye, such as antibiotic drops and medications to treat glaucoma, can be more precisely administered with less waste, which could save a subject a significant amount of money during the course of treatment.

Embodiments of the invention may include a volume of liquid to administer a plurality of drops. For example, in an embodiment, the internal cavity of the eyedropper may range from about 5 milliliters to about 50 milliliters of liquid, or in another embodiment, the internal cavity may range from about 5 milliters to about 30 milliters, or in another embodiment, the internal cavity may range from about 7 milliliters to about 15 milliliters. In another embodiment, the internal cavity includes a volume of liquid sufficient for a single use, such as about 50 microliters or less; or in another embodiment, about 25 microliters or less; or in another embodiment, the internal cavity includes a volume of liquid in a range from about 5 microliters to about 25 microliters. Preferably, the internal cavity includes a volume in a range of about 15 microliters or less, or in a range of about 5 microliters to about 15 microliters. In the single use embodiments, a plurality of eyedroppers may be packaged together, such as with breakaway structures, wherein each eyedropper includes a volume of liquid sufficient for a single administration of liquid onto the surface of the eye.

Embodiments of the invention may be made using standard blow molding techniques used with thermoplastic materials. The body 4 of the eyedropper 2 is made of a material having sufficient flexibility to allow the user to increase the internal pressure in the body 4 to expel a drop of liquid from the nozzle 27 while providing enough support to the body 4 so that the eyedropper 2 is stabilized when the first and second feet 14, 16 are in contact with the subject's face 32. The body 4 and nozzle 27 may be made from materials deemed fit by the U.S. Food and Drug Administration for the administration of eye drops. In an embodiment, the body 4 and nozzle 27 are made of a plastic material such as polyethylene or polypropylene. In another embodiment, the body 4 is made from a low-density polyethylene and the nozzle is made from a high-density polyethylene. The nozzle 27 may be made from the same material as the body 4 or may be made from a different material. The nozzle 27 may be less flexible than the body 4. The nozzle 27 may be co-molded with the body 4 or formed separately. In an exemplary method, the body 4 is formed by blow molding and the nozzle 27 is formed in a separate operation. In an embodiment, a nozzle portion 75 extending from the reservoir body 18 is inserted into the flange 74 the nozzle 27. In this embodiment, a cap 58 is received on threads 72 onto the outer surface 76 of the nozzle flange 74 of the nozzle 27. This embodiment is especially helpful when the body of the bottle if made from particularly flexible materials, such as flexibility, because the flexibility of the reservoir body 18 of the eyedropper 2 may not form a tight seal between the reservoir body 18 and the nozzle 27 when the nozzle 27 is instead inserted into the reservoir body 18. Inserting the body portion into the nozzle remedies the leaking problem. In another embodiment, the nozzle 27 is then inserted into an opening in the reservoir body 18 formed to accept the nozzle 27 and the outer surface of the portion of the reservoir body 18 that accepts the nozzle 27 may be threaded 72 to receive a cap 58. For embodiments in which the nozzle 27 is formed separately from the body, it may be joined to the body with known methods, such as with a friction fit, using an adhesive, or by welding. Preferably, the base 54 of the nozzle 27 does not project above or interfere with the operation of the funnel-shaped portion 59.

While the exemplary embodiments of the invention described herein are shown having an arched crescent-shaped structure, the function of stabilizing the eyedropper 2 by contacting the face 32 on opposite sides of the eye socket 34 with the first and second feet 14, 16 during administration of drops may be accomplished with non-arching structures. For example, reservoir body 18 could have legs 6, 8 of varying shapes such as straight legs, curved legs, bent legs and combinations thereof so long as the legs 6, 8 allow the feet to contact the subject's face 32 on opposite sides of the eye socket 34 and that the distance d between the feet 14, 16 is greater than or equal to height h of the eyedropper 2.

While the exemplary embodiments shown herein are illustrated as having two feet, additional feet could be utilized. For example, a third or fourth leg having a third or fourth foot could be utilized. Moreover, one or both of the first and second legs could be configured to accommodate a third or fourth foot, such as by splitting the leg along a portion of its length near the feet.

In use, the first foot 14 contacts a first area 30 on the subject's face below or lateral to the eye socket 34 such as on or near the cheek 40 or the temporal area, and a second area 38 on the opposite side of the eye socket 34 such as on or near the eyebrow 44 or the nose, thereby creating a stable situation to administer the drops onto the surface of the eye 57 (FIG. 1). The hand administering the drop no longer waves freely above the eye 57 (like with current eyedroppers) but is instead stabilized by the first and second feet contacting the face on opposite sides of the eye socket. Additional stability is provided by the height h of the eyedropper 2 relative to the distance d between the first and second feet 14, 16 which results in the eyedropper 2 being relatively close to the face 32. The relative short height h compared to the distance d between the two feet 14, 16 allows the user to contact his or her face 32 with other parts of his or her hand, such as his other fingers or the outer edges of the palm, to act as an additional point of stability. The stability control conferred by the current structure is a major advantage not provided by eyedroppers previously available. In addition, in embodiments in which the nozzle 27 is positioned closer to the second foot 16, than the first foot 14, head tilt required when administering drops onto the surface of the eye 57 is reduced while also decreasing distance of drop fall. If desired, the structure of the eyedropper 2 may assist the user in holding the eye 57 open by applying a downward pressure on the eyedropper 2 after contacting the face 32 below the eye 57 with the first foot 14 before contacting the face 32 above the eye 57. To assist with holding the eye open, the surface of the first foot 14 may include structures, such as a plurality of raised projections 80, to improve grip of the foot 14 on the skin of the face (FIG. 2). This all happens in a matter of seconds.

FIGS. 4 and 4A illustrates an exemplary embodiment of the invention for administering a liquid drop 88 having a volume of about 0.02 cc. In this illustrative embodiment, the first and second contact surfaces 26A and 26B have a diameter of about 4 mm and are spaced apart from one another by a distance of about 2 mm. The internal volume that is displaced when the first and second contact surfaces 26A and 26B are made to contact each other is estimated to be about 0.026 cc. The nozzle 27 in the illustrative embodiment has an angled channel 90 with an opening 92 on the internal cavity side of about 0.4 mm. The channel 90 has a length of about 10.5 mm and opens at an angle of about 6 degrees.

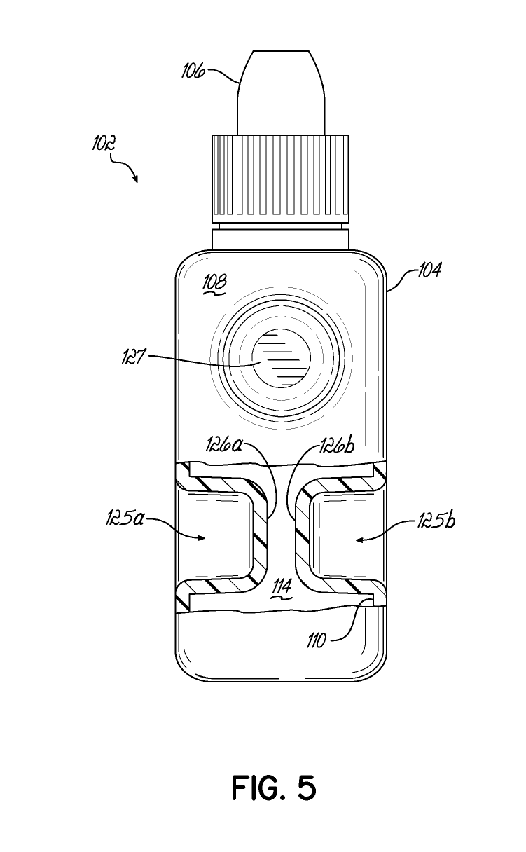

FIG. 5 illustrates an alternative embodiment of the invention directed to the use of pairs of raised projections with a traditionally shaped bottle. The bottle 102 has a standard shaped body 104 with a nozzle and cap 106 at one end thereof. The body 104 also includes an outer surface 108 and an inner surface 110 defining a portion of an internal cavity 114. The inner surface 110 of the body 104 also includes a first projecting portion 125a and an oppositely disposed and spaced apart second projecting portion 125b extending into the internal cavity 114. The first and second projecting portions 125a, 125b each have a contact surface 126a, 126b. The body also includes a second pair of projecting portions 127 that may be result in a different displaced volume from the internal cavity 114. The second pair of projecting portions 127 may be useful if the displaced volume of the internal cavity necessary to dispense the desired volume from the nozzle changes as the volume of liquid in the internal cavity changes. The second pair of projection portions 127 may also be useful to dispense a second predetermined volume of liquid from the nozzle. The projecting portions 125a, 125b, and 127 operate on the same principles as described above.

Embodiments of the invention are designed for on target performance, creating a high rate of success getting the desired liquid into the eye and can alleviate anxiety during administration of drops. Embodiments of the invention allow “on the go” application of drops. Embodiments of the invention also decrease the likelihood of contaminating the tip of the nozzle because the position of the nozzle relative to the first and second feet prevents the nozzle from contacting with the eye or surrounding tissue during use. Embodiments of the invention can also be used by children, teens, adults, and elderly with great ease.

While the present invention has been illustrated by the description of specific embodiments thereof, and while the embodiments have been described in considerable detail, it is not intended to restrict or in any way limit the scope of the appended claims to such detail. For example, the illustrative embodiments include first and second projecting portions with oppositely disposed first and second contact surfaces. An alternative embodiment may include a first projecting portion with a first contact surface oppositely disposed and spaced apart from a second contact surface that is not on a projecting portion but is instead on a non-projecting portion of the inner surface of the internal cavity of the eyedropper. The first and second contact surfaces of this alternative embodiment operate on the same principles as described above.

The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

1. An eyedropper for administering a volume of liquid onto the surface of an eye, the eyedropper comprising:
a body having a reservoir body with an inner surface defining an internal cavity, and a nozzle in fluid communication with the internal cavity and wherein the inner surface has a first contact surface on a first projecting portion with and a second contact surface wherein the first contact surface is positioned opposite to and spaced apart from the second contact surface and the first and second contact surfaces are configured to limit the displacement of the volume of the internal cavity when the body is squeezed to an amount sufficient to expel a predetermined volume of liquid from the nozzle.

2. The eyedropper of claim 1 wherein the predetermined volume of liquid is one drop.

3. The eyedropper of claim 1 wherein body further comprises a first leg with a first foot located at the free end of the first leg and a second leg with a second foot located at the free end of the second leg, the first leg having a length and the second leg having a length, the length of the first leg is greater than the length of the second leg.

4. The eyedropper of claim 3 wherein the distance between the first and second feet is greater than the height of the eyedropper.

5. The eyedropper of claim 3 wherein the distance between the first foot and the second foot is such that the first foot contacts the face of a subject adjacent one side of an eye socket and the second foot contacts the face of the subject on the opposite side of the eye socket from the first foot.

6. The eyedropper of claim 3 wherein the first foot is configured to contact the face of a subject below the eye socket and the second foot is configured to contact the face above the eye socket.

7. The eyedropper of claim 3 wherein the first foot is configured to contact the face of the subject lateral to the eye socket and the second foot is configured to contact the face of the subject medial to the eye socket.

8. The eyedropper of claim 3 wherein the distance between the first and second feet is in a range from about 2.5 inches to about 4.5 inches.

9. The eyedropper of claim 3 wherein the height is in a range from about inches 1.5 to about 2.5 inches.

10. The eyedropper of claim 1 wherein the eyedropper includes a reservoir portion configured to receive a volume of liquid for administering to the surface of the eye of the subject.

11. The eyedropper of claim 10 wherein the volume is in a range of about 5 microliters and about 50 microliters.

12. The eyedropper of claim 3 wherein the nozzle does not extend beyond a plane that is tangential to both the first and second feet.

13. The eyedropper of claim 3 wherein the first leg has a first axis and the second leg has a second axis and the intersection of the first axis with the second axis at a point on the outer surface of the body directly opposite the nozzle forms an angle facing the nozzle that is less than about 180 degrees.

14. The eyedropper of claim 13 wherein the angle is between about 45 degrees and about 175 degrees.

15. The eyedropper of claim 3 wherein at least one of the first foot or the second foot includes a structure configured to improve the grip of the foot on the skin of the face of a subject.

16. The eyedropper of claim 15 wherein the structure is a plurality of raised projections.

17. The eyedropper of claim 3 further comprising a drop stopper adjacent the nozzle configured to direct liquid in the internal cavity toward the nozzle.

18. The eyedropper of claim 1 further comprising a second projecting portion wherein the second contact surface is on the second projecting portion.

19. The eyedropper of claim 1 wherein the volume of the internal cavity that is displaced when the body is squeezed is about 15% to about 75% greater than the predetermined volume of the liquid dispensed from the nozzle.

20. A method for administering liquid drops onto the surface of the eye comprising:
providing an eyedropper in accordance with any previous claim;
squeezing an outer surface of the eyedropper to cause a first contact surface to contact a second contact surface to displace a volume of the internal cavity of the eyedropper in an amount sufficient to expel a predetermined volume of liquid from a nozzle on the reservoir body.

21. The method of claim 20 further comprising tilting the eyedropper in a first direction to fill a nozzle reservoir with a volume of liquid; and
tilting the eyedropper in a second direction to collect liquid not in the nozzle reservoir in a body reservoir prior to expelling the volume of liquid.

22. An eyedropper comprising a reservoir body and a nozzle, wherein the reservoir body has a nozzle portion projecting therefrom and the nozzle has a base with a tip projecting from one side and a flange projecting from the opposite side, wherein the flange receives the nozzle portion.

23. The eyedropper of claim 22 wherein the nozzle portion has an outer surface with one or more annular rings.

24. The eyedropper of claim 22 where the flange further includes an outer surface with a thread.
